(19) United States
(12) Patent Application Publication
(10) Pub. No.: US 2008/0103487 A1
(43) Pub. Date: May 1, 2008

(54) Connection Structure of a Transfusion Line

(76) Inventor: Susumu Miyasaka, Tokyo (JP)

Correspondence Address: TYCO HEALTHCARE - EDWARD S. JARMOLOWICZ, 15 HAMPSHIRE STREET, MANSFIELD, MA, 02048, US

(21) Appl. No.: 11/576,876

(22) PCT Filed: Oct. 6, 2005
(86) PCT No.: PCT/EP05/10773

§ 371 Date: Dec. 13, 2007

(30) Foreign Application Priority Data
Oct. 6, 2004 (JP) 2004294257

(51) Int. Cl. A61M 39/10

(52) U.S. Cl. 604/537

(57) ABSTRACT

A connection structure of a transfusion line includes: an injection tube including a bottomed tubular injection part having an opening in the leading end side circumferential wall, and an introduction part communicating with the inside of the injection part; a bottomed outer tube part having a bottom portion linked to the injection tube part and opened to the leading end side for fitting a mating member therein; and a protective shutter externally inserted on the injection part for closing the opening and adapted to move along the axis of the injection part, when it receives a pressing force not lower than a predetermined level toward the base portion side, thereby to open the opening, and to return to the leading end side, when the pressing force is released, thereby to close the opening.

### CROSS-REFERENCE TO RELATED APPLICATIONS

This application is based on and claims the benefit of International (PCT) Application Serial No. PCT/EP2005/010773, filed Oct. 6, 2005, which claims priority from Japanese Patent Application No. 2004-294257, filed Oct. 6, 2004, both of which are herein incorporated by reference in their entirety.

### FIELD OF THE INVENTION

The present invention relates to a connection structure of a transfusion line (or other medical liquid flow line).

### BACKGROUND OF THE INVENTION

In the transfusion line of a medical tool, there is used the closed system which has an infection preventing function and a needle piercing accident preventing function. For example, the closed system is provided with a septum or the like on the side of a female connector. FIG. 11 is a longitudinal section showing one example of a medical joint part of the prior art, which is composed of a male connector and a female connector. The male connector 30 is provided with a male Luer 31, and the female connector 40 has a septum 42 arranged in a hollow retainer 41 for fitting the male Luer 31 therein. The retainer 41 is provided with a bulging portion 53 bulging outward from the outer circumference of the retainer 41 for being fastened by the screw of a fastening tube part 32 of the male connector 30. As a result, the male connector 30 and the female connector 40 are fixed by causing them to engage with each other.

JP-T-8-500983 (page 2, FIG. 2) shows one example of such a device. The medical joint portion of the prior art is provided with an opening 33 at the leading end of the male Luer 31 of the male connector 30. After a priming, however, a liquid leaks from the opening 33, and the liquid leaks in the effluent state to invite an unclean state so that the opening 33 is not sufficiently protected in its vicinity.

### SUMMARY OF THE INVENTION

In one aspect of the present invention, a connection structure of a transfusion line having a protective shutter for passing a medical liquid therethrough comprises an injection tube including a bottomed tubular injection part having an opening in the leading end side circumferential wall, and an introduction part communicating with the inside of said injection part. A bottomed outer tube part has a bottom portion linked to the injection tube part and is opened to the leading end side for fitting a mating partner therein. A protective shutter externally inserted on the injection part closes the opening. The shutter is adapted to move along the axis of the injection part when it receives a pressing force not lower than a predetermined level toward the base portion side to open the opening. The shutter is adapted to return to the leading end side when the pressing force is released to close the opening.

In another aspect, a connector for a medical liquid flow line comprises an injection tube with a closed leading end and a tube wall with a luminal surface and an abluminal surface. There is at least one through hole extending through the tube wall near the closed leading end. A shutter slides on the abluminal surface and occludes the at least one through hole when in a closure disposition. The shutter is displaceable by sliding on the abluminal surface to an open disposition when the shutter is subject to a pressing force when the connector is connected within the flow line. The shutter is biased to the closure disposition so that when the connector is disconnected from the flow line, the shutter reverts to the closure disposition.

In yet another aspect, a connector comprises a male connector. The male connector includes an injection tube including a bottomed tubular injection part having an opening in the leading end side circumferential wall, and an introduction part communicating with the inside of the injection part. A bottomed outer tube part has a bottom portion linked to the injection tube part and opened to the leading end side for fitting a mating partner therein. A protective shutter externally inserted on the injection part closes the opening. The shutter is adapted to move along the axis of the injection part when it receives a pressing force not lower than a predetermined level toward the base portion side to open the opening. The shutter is adapted to return to the leading end side when the pressing force is released to close the opening. The connector comprises a female connector including an outer tube having a fitted portion to be fitted in the outer tube part. A septum is arranged on the inner circumference side of the outer tube and is made of an elastic member having a slit formed for inserting the injection part. The female connector includes a fixing means for fixing the septum in the outer tube part and a space adjoining the septum that is capable of opening the injection part leading end containing the bore portion. An inner tube is formed on the inner circumference side of the outer tube part for communicating from the space to the outside. A connection means is connected to a discharge side member, into which the liquid discharged from the inner tube flows.

### BRIEF DESCRIPTION OF THE DRAWINGS

FIG. 1a is a longitudinal section of a male connector according to Embodiment 1 of the invention.

FIG. 1b is a front elevation of the male connector taken along arrow A of FIG. 1a.

FIG. 2a is a longitudinal section of a female connector to be fitted on the male connector of FIG. 1.

FIG. 2b is a front elevation of the female connector taken along arrow B of FIG. 2a.

FIG. 3a is a longitudinal section of the state, in which a protective cap is fitted on the male connector 1 of Embodiment 1.

FIG. 3b is a front elevation of the state, in which the protective cap is fitted on the male connector 1, as taken in arrow C of FIG. 3a.

FIG. 4 is a longitudinal section of the state, in which the male connector and the female connector of Embodiment 1 are jointed to each other.

FIG. 5 is a longitudinal section of a male connector according to Embodiment 2 of the invention.

FIG. 6 is a longitudinal section of the state, in which the male connector and the female connector of Embodiment 2 are jointed to each other.

FIG. 7 is a longitudinal section of a male connector according to Embodiment 3 of the invention.

FIG. 8 is a longitudinal section of the state, in which the opening 5 of the male connector of Embodiment 3 is opened.

FIG. 9 is a longitudinal section of a male connector according to Embodiment 4 of the invention.

FIG. 10 is a longitudinal section of the state, in which the opening 5 of the male connector of Embodiment 4 is opened.

FIG. 11 is a longitudinal section showing one example of a medical coupling device of the prior art.

### DETAILED DESCRIPTION OF THE DRAWINGS

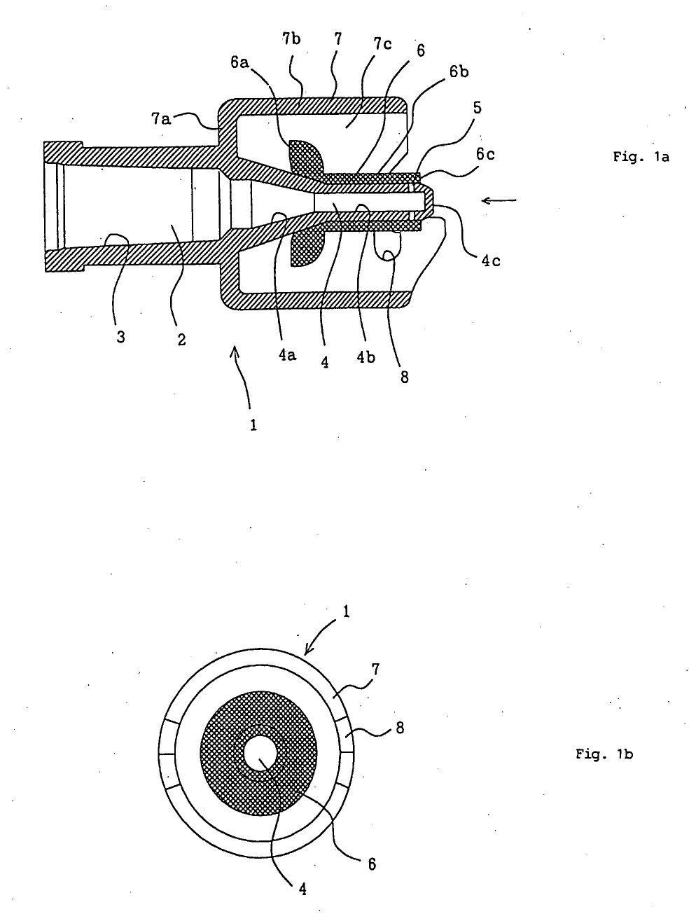

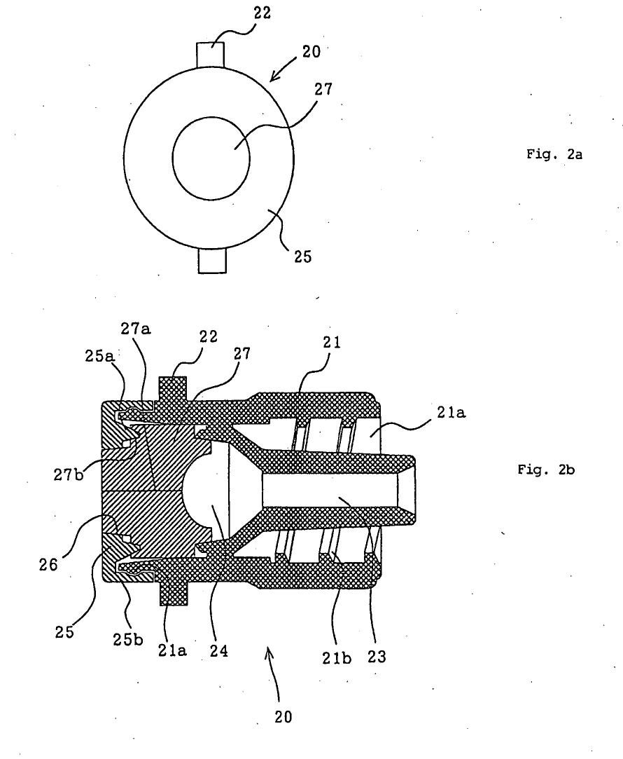

FIG. 1 is a longitudinal section of a male connector according to Embodiment 1 of the invention, and FIG. 2 is a longitudinal section of a female connector to be fitted in the male connector. A male connector 1 is formed of plastics or the like and attached to a tube or syringe (although not shown), and is constructed in its entirety into a tube, as shown in FIG. 1b). A female connector 20, as shown in FIG. 2, is formed of plastics or the like and attached to the port of a three-way plug or the hub of a catheter (although not shown), and is formed in its entirety into a tube, as shown in FIG. 2b). Moreover, the male connector 1 is fitted in the female connector 20 to construct a medical joint part.

As shown in FIG. 1, the male connector 1 is provided with an injection tube 2 for injecting a liquid such as a medical liquid. The injection tube 2 is constructed to include a cylindrical introduction part 3 positioned on the base portion side (or the upstream side) and connected to the tube for injecting the liquid such as the medical liquid, and an injection part 4 positioned on the leading end portion side (or the downstream side) and more diametrically reduced than the introduction part 3. Moreover, the injection part 4 is composed of a taper injection portion 4a positioned on the side of the introduction part 3 and having a frusto-conical shape gradually diametrically reduced toward the leading end portion side, and a generally bottomed cylindrical leading end injection portion 4b positioned on the leading end portion side of the taper injection portion 4a and having its leading end closed with a leading end wall 4c and its leading end portion side slightly gradually diametrically reduced. In the leading end side wall of the leading end injection portion 4b, there is formed an opening 5 for providing communication between the injection tube 2 and the inner and outer circumferences of the injection part 4.

From the vicinity of the leading end portion of the leading end injection portion 4b to the leading end portion side of the taper injection portion 4a, a protective shutter 6 formed of an elastic member such as rubber into a tubular shape for protecting the vicinity of the leading end portion of the injection part 4 against invasion of bacteria or an impurity is externally inserted axially slidably along the outer circumference wall of the injection part 4. The opening 5 of the injection part 4 is opened/closed as that protective shutter 6 slides. This protective shutter 6 is diametrically enlarged on its base portion side to form a stopper portion 6a and has its internal diameter tapered along the outer wall taper face of the taper injection portion 4a and its leading end portion side formed into a cylindrical portion 6b to form an abutment face 6c at its leading end portion and into a cylindrical shape along the outer wall face of the leading end injection portion 4b.

Moreover, the taper injection portion 4a enlarges the diameter of the protective shutter 6 elastically with the taper face diametrically enlarged on the base portion side, when the protective shutter 6 is retracted toward the base portion to open the opening 5 of the injection part 4 by a pressing force at a predetermined level or higher, so that a biasing force in the forward direction, i.e., toward the leading end portion side is applied to the protective shutter 6 by the restoring elasticity of the protective shutter 6 in the diametrically reducing direction. Here, the predetermined pressing force can be arbitrarily set by considering the frictional force between the protective shutter 6 and the injection part 4 and the taper angle on the base portion side of the injection part 4. In case, moreover, the protective shutter 6 is returned to the leading end portion side by the biasing force in the forward direction, the elastic force of the protective shutter 6 is so adjusted at the fabrication time as to stop at the initial position.

From the vicinity of the base portion to the leading end portion side of the taper injection portion 4a of the injection part 4, on the other hand, there is formed a bottomed cylindrical outer tube part 7, which has a bottom portion 7a on the base portion side, a circumferential wall 7b on the leading end portion side and a space portion 7c in the inside, and which is opened on the leading end portion side. In the leading end portion side of the circumferential wall 7b, there is formed a retaining hole 8 for fitting a retained portion 22 therein.

As shown in FIG. 2, the female connector 20 is provided with an outer tube part 21 having a helical line 21b in an inner wall 21a on its base portion side (or the downstream side), so that the outer tube part 21 is screwed in the port of a three-way plug by that helical line 21b. Moreover, the outer tube part 21 is provided, on the outer wall of its leading end portion side (or the upstream side), with the retained portion 22 to engage with the retaining hole 8 of the male connector 1. The outer tube part 21 is further provided, on the base portion side of the inner wall 21a than the leading end portion side, with an injection tube 23 having a generally Y-shaped section. A space portion 24 is formed on the leading end portion side of the injection tube 23. In the opening positioned on the leading end of the outer tube part 21, there is disposed a holding portion 25 having a generally C-shaped section for closing the opening. The holding portion 25 is retained such that a recess 25b formed in the side face inner wall engages with a bulging portion 21a formed on the leading end portion side outer wall of the outer tube part 21. A circular bore portion 26 is formed in the vicinity of the center of the holding portion 25, and a septum 27 made of an elastic member is filled from the circular bore portion 26 to the vicinity of the central portion of the space portion 24 and is retained by holding an annular ridge 25a of the holding portion 25 in engagement with an annular groove portion 27b of the septum 27. Here, the septum 27 is provided with a slit 27a for fitting the injection part 4 of the male connector 1.

When the male connector 1 is fitted in the female connector 20, the leading end injection portion 4b of the injection part 4 of the male connector 1 is inserted in a sealed state into the septum 27 of the female connector 20. At this time, the abutment face 6c positioned at the leading end portion of the protective shutter 6 comes into abutment against the surface of the septum 27, and the protective shutter 6 retracts to the base portion side so that the opening 5 can be opened to inject the liquid from the side of the male connector 1 to the side of the female connector 20.

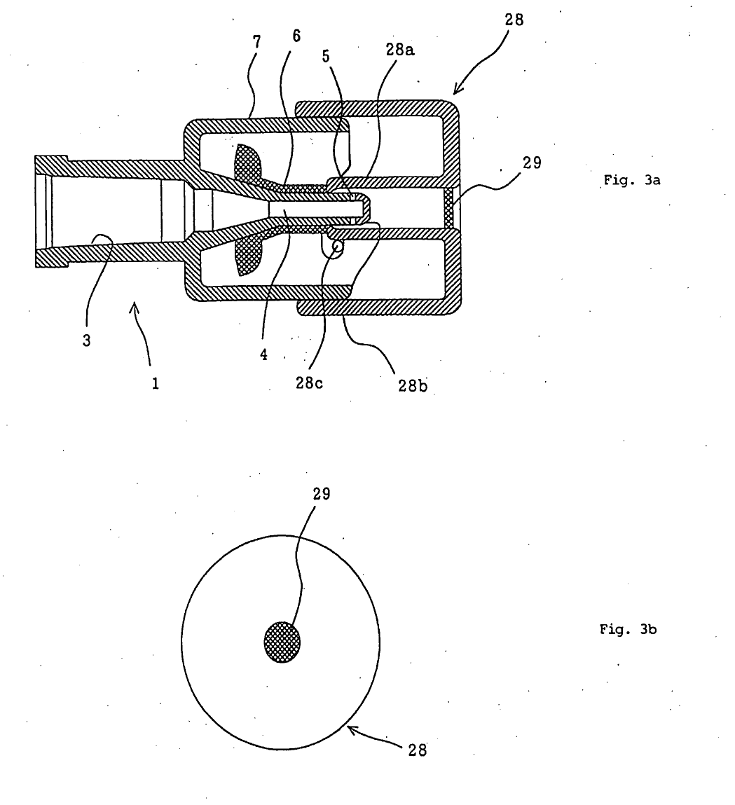

The actions of Embodiment 1 thus constructed are described. First of all, a protective cap 28 is fitted before use on the male connector T1 as shown in FIG. 3. The protective cap 28 is provided with a cylindrical enclosure portion 28a for covering the injection part 4 of the male connector 1, and a cylindrical outer tube enclosure portion 28b for enclosing the leading end portion side of the outer tube part 7 of the male connector 1. The enclosure portion 28a is provided, at the end portion of the opposite injection part side, with a filter 29 for passing air therethrough only from the inside to the outside but not the liquid. The male connector 1 is so fitted in the protective cap 28 that a retained portion 28c formed in the inner circumference of the outer tube enclosure portion 28b of the protective cap 28 is made, like the case of the female connector 20, to engage with the retaining hole 8 of the male connector 1. In this state, the introduction part 3 and the injection part 4 of the male connector 1 and the inner circumference of the protective cap enclosure portion 28a are filled with the medical liquid. Thus, merely by removing the protective cap 28, the opening 5 of the injection part 4 is closed with the protective shutter 6 so that the priming can be completed.

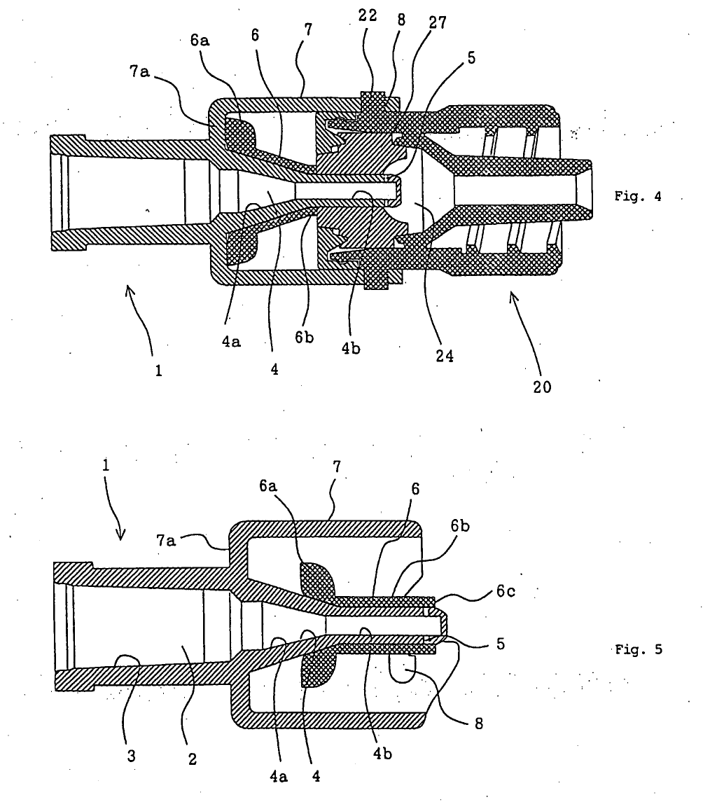

After this priming operation, the leading end portion of the injection part 4 of the male connector 1 is inserted from the slit 27a of the septum 27 of the female connector 20, as located on the three-way plug side, and the male connector 1 is turned. Then, the retained portion 22 of the female connector 20 is retained in and linked to the retaining hole 8 of the male connector 1, as shown in FIG. 4, so that the portion of the opening 5 of the leading end injection portion 4b of the male connector 1 is introduced into the space portion 24 of the female connector 20. At this time, the protective shutter 6 of the male connector 1 is pressed to the base portion side of the injection part 4 by the surface of the septum 27 of the female connector 20 and is retracted toward the base portion side along the axial direction of the injection part 4 so that the opening 5 of the protective shutter 6 is opened. The protective shutter 6 further retracts and stops when its stopper portion 6a abuts against the bottom portion 7a of the outer tube part 7.

Thus, the protective shutter 6 of the male connector 1 is compressed by the septum 27 of the female connector 20 so that the opening 5 of the male connector 1 is opened not in the atmosphere but only in the space portion 24. Moreover, the injection part 4 of the male connector 1 is inserted into the inside of the female connector 20 so that the opening 5 of the injection part 4 is positioned in the space portion 24. As a result, the male connector 1 and the female connector 20 can communicate with each other through the opening 5 of the injection part 4 thereby to pass the liquid. In case the male connector 1 is disconnected when it is to be connected to the female connector 20, the protective shutter 6 is returned to the leading end portion side by its elastic force when the male connector 1 is disconnected, so that the opening 5 of the male connector 1 is closed to block the passage of the liquid.

At the time of the communication between the male connector 1 and the female connector 20, the protective shutter 6 has been retracted and diametrically enlarged to the diametrically larger side of the taper injection portion 4a. As a result, the protective shutter 6 establishes an elasticity to return to the diametrically smaller side thereby to establish a force to move to the diametrically smaller side of the taper injection portion 4a, i.e., a biasing force toward the leading end portion side. When the injection part 4 of the male connector 1 is extracted from the septum 27 of the female connector 20, the protective shutter 6 is slid to the diametrically smaller side along the taper face of the taper injection portion 4a by the restoring force to the diametrically reduced side, so that the opening 5 of the leading end injection portion 4b is occluded by the protective shutter 6.

According to Embodiment 1, the biasing force to the leading end portion side can be applied to the protective shutter 6 by forming the taper injection portion 4a in the male connector 1 and by externally inserting the protective shutter of the elastic member on the taper injection portion 4a. Thus, the opening 5 of the male connector 1 is normally closed by the protective shutter 6 and is opened, only when needed, by applying the force to the base portion side. As a result, the leakage of the liquid at the releasing time of the connector or at the priming time can be suppressed to suppress its diffusion, which would otherwise cause the infection.

FIG. 5 is a longitudinal section of a male connector according to Embodiment 2 of the invention. In Embodiment 2, the cylindrical portion 6b of the protective shutter 6 is made of a deformable elastic member. The remaining construction is substantially similar to that of the case exemplified in Embodiment 1, and its description is omitted.

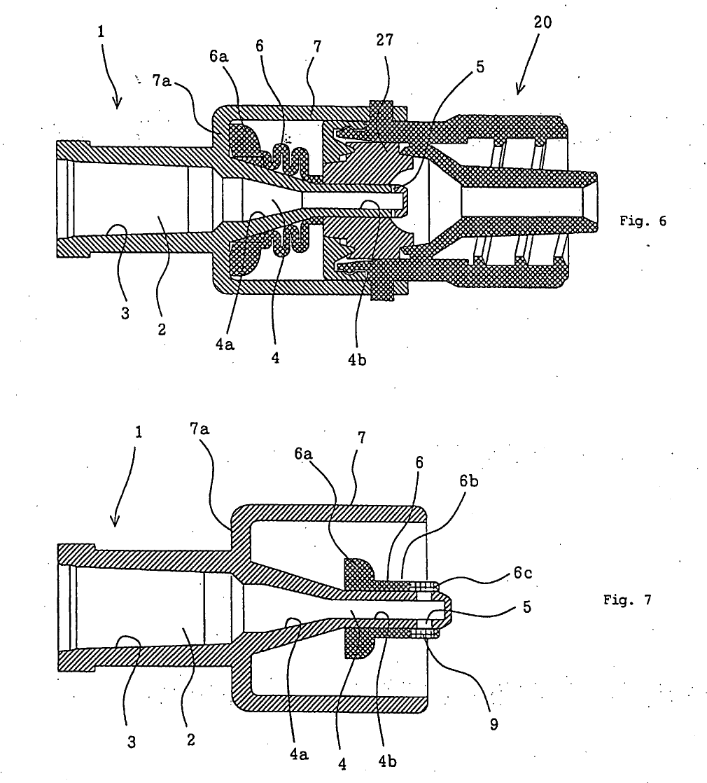

The actions of Embodiment 2 thus constructed are described. When the male connector 1 shown in FIG. 5 is inserted into the female connector 20, the protective shutter 6 is pressed by the surface of the septum 27 to slide along the axial direction of the injection part 4 to the base portion side, as shown in FIG. 6, so that its stopper portion βa comes into abutment against the bottom portion 7a of the outer tube part 7. When the protective shutter 6 is pressed further by the surface of the septum 26, it is deformed into a corrugated shape.

At this time, the protective shutter 6 is diametrically-enlarged by the taper injection portion 4a and is compressed into the corrugated shape. As a result, the restoring elasticity in the diametrically reducing direction occurs in the protective shutter 6 thereby to establish the force to move to the diametrically smaller side of the taper injection portion 4a, i.e., the biasing force to the leading end portion side. When the male connector 1 is then extracted from the female connector 20, the protective shutter 6 is slid by the restoring force to the diametrically smaller side, i.e., to the leading end portion side along the taper face of the taper injection portion 4a, so that the opening 5 of the injection part 4 is clogged by the protective shutter 6 thereby to restore the state, as shown in FIG. 5. The remaining actions and advantages are substantially similar to those of the case exemplified in Embodiment 1, and their description is omitted.

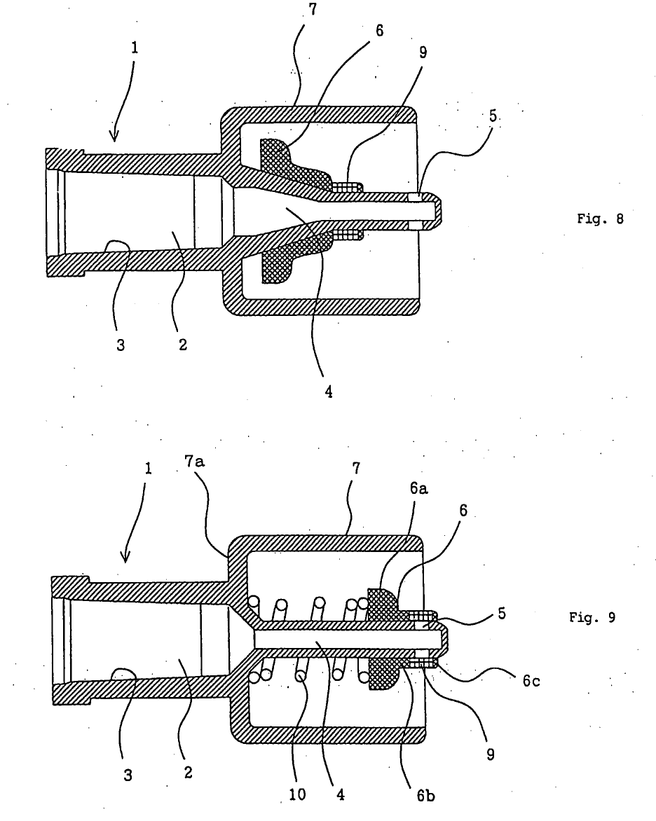

FIG. 7 is a longitudinal section of a male connector according to Embodiment 3 of the invention. In Embodiment 3, the protective shutter 6, as exemplified in Embodiment 1, is provided with an air-bleeding filter. In order to protect the vicinity of the opening 5, the protective shutter 6 formed of an elastic member such as rubber into a tubular shape is so externally fitted on the leading end injection portion 4b as to slide in the axial direction along the outer circumference wall of the injection part 4. As the protective shutter 6 slides, the opening 5 of the injection part 4 is freely opened/closed, as shown in FIG. 8. This protective shutter 6 is provided, in the vicinity of its leading end portion and at the portion corresponding to the opening 5, with a filter for covering the opening 5 widely to bleed the air. This filter 9 is made of a member for passing the air therethrough but not a liquid such as the medical liquid. The remaining actions and advantages are substantially similar to those of the case exemplified in Embodiment 1, and their description is omitted.

In addition to the advantages of Embodiment 1, according to Embodiment 3, the protective shutter 6 is provided at its portion with the air-bleeding filter 9. After the priming, therefore, even when the opening 5 is closed by the protective shutter 6, the residual air can be further discharged while preventing the leakage of the medical liquid from the opening 5. The protective shutter 6, as exemplified in Embodiment 2 with a construction similar to that of this embodiment, may also be provided with the air-bleeding filter. This filter has actions substantially similar to those of this embodiment.

FIG. 9 is a longitudinal section of a male connector according to Embodiment 4 of the invention. In Embodiment 4, the protective shutter 6 is provided on its base portion side with an elastic member such as a coil spring and at its portion with the filter 9 for bleeding the air. As shown in FIG. 9, the injection tube 2 of the male connector 1 is constructed to include the cylindrical introduction part 3 positioned on the base portion side and connected to the tube, and the bottomed tubular injection part 4 positioned on the leading end portion side and diametrically reduced more than the introduction part 3. In the side wall on the leading end portion side of the injection part 4, the opening 5 is formed to provide the communication between the injection tube 2 and the outside.

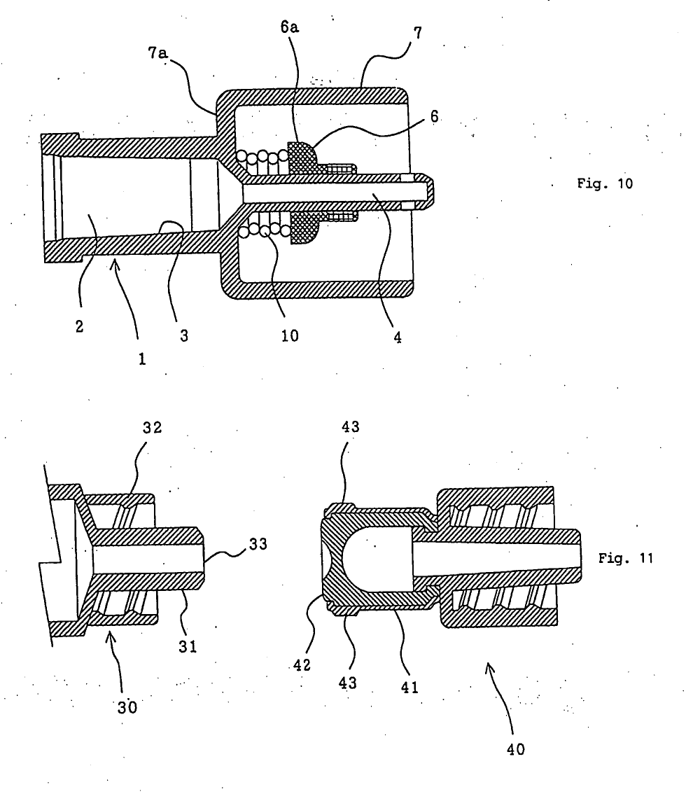

In order to protect the vicinity of the leading end portion of the injection part 4, the protective shutter 6 formed of an elastic member such as rubber into a tubular shape is externally fitted slidably in the axial direction along the outer circumference wall of the injection part 4 so that the opening 5 of the injection part 4 is freely opened/closed as the protective shutter 6 slides. This protective shutter 6 is diametrically enlarged on its base portion side to form the stopper portion 6a and is provided with the cylindrical portion 6b on the leading end portion side and the abutment face 6c at its leading end portion. On the outer circumference of the injection part 4 positioned on the base portion side of the protective shutter 6, a coil spring 10 is arranged to have its one end retained on the stopper portion 6a of the protective shutter 6 and its other end retained on the bottom portion 7a of the outer tube part 7. Here, the elastic force of the coil spring 10 is so adjusted that the protective shutter 6 may stop at the initial position in case it is returned from the position shown in FIG. 10 to the position on the leading end portion side, as shown in FIG. 9, by the biasing force in the forward direction.

The protective shutter 6 is provided, in the vicinity of its leading end portion and at the portion corresponding to the opening 5, with the filter 9 for covering the opening 5 widely to bleed the air. This filter 9 is made of a member for passing the air but not the liquid such as the medical liquid therethrough. The remaining construction and actions are substantially-similar to those of Embodiment 1, and their description is omitted. According to Embodiment 4, in addition to the effects of Embodiment 1, the coil spring 10 is mounted on the base portion side of the protective shutter 6 so that the reliable biasing force to the leading end portion side can be obtained. At the same time, the air-bleeding filter 9 is mounted in the portion of the protective shutter 6 so that the residual air can be further removed while preventing the leakage of the medical liquid from the opening 5 even when the opening 5 is closed by the protective shutter 6 after the priming.

The shapes of the connectors exemplified in the foregoing individual embodiments can also be so applied to a syringe that the shape of the leading end portion of the syringe may be shaped into the injection part of the male connector. Moreover, the shape of the Luer tip or the catheter tip may also be applied to the shape of the injection part.

At least one embodiment of the present invention provides a connection structure of a transfusion line, which can be attached to and detached from the transfusion line without any leakage from the opening when the connected state is released, and a connector having the connection structure.

According to at least one embodiment of the invention, there is provided a connection structure of a transfusion line for passing a medical liquid therethrough, which comprises: an injection tube including a bottomed tubular injection part having an opening in the leading end side circumferential wall, and an introduction part communicating with the inside of the injection part; a bottomed outer tube part having a bottom portion linked to the injection tube part and opened to the leading end side for fitting a mating partner therein; and a protective shutter externally inserted on the injection part for closing the opening and adapted to move along the axis of the injection part, when it receives a pressing force not lower than a predetermined level toward the base portion side, thereby to open the opening, and to return to the leading end side, when the pressing force is released, thereby to close the opening. Here, the connection structure for the transfusion line may be exemplified by the connection structure having the connector constructed to include the male connector and the female connector, as will be described hereinafter. In this case, the aforementioned mating partner is the female connector. Moreover, the aforementioned pressing force not lower than a predetermined level implies a pressing force at a level capable of opening the opening when the> protective shutter is pressed to the injection part base portion side.

According to at least one embodiment of the invention, moreover, in the connection structure of the transfusion line for passing a medical liquid therethrough, the injection part is provided on its base portion side with a taper portion diametrically reduced in the direction to the leading end portion so that the protective shutter is diametrically enlarged along the taper portion, when it slides to the taper portion side, to establish a biasing force toward the leading end portion side.

According to at least one embodiment of the invention, moreover, in the connection structure of the transfusion line for passing a medical liquid therethrough, the injection part is provided on its base portion side with a taper portion diametrically reduced in the direction to the leading end portion so that the protective shutter is diametrically enlarged along the taper portion and compressed to the base portion side in the axial direction, when it slides to the taper portion side, to establish a biasing force toward the leading end portion side.

According to at least one embodiment of the invention, moreover, in the connection structure of the transfusion line for passing a medical liquid therethrough, the injection part is provided with a spring portion for biasing the protective shutter to the leading end portion side, so that it is biased to the leading end portion side by the spring portion when the protective shutter slides to the spring portion side.

According to at least one embodiment of the invention, moreover, in the connection structure of the transfusion line for passing a medical liquid therethrough, the protective shutter is provided with an air-bleeding filter in front of the opening.

According to at least one embodiment of the invention, moreover, in the connection structure of the transfusion line for passing a medical liquid therethrough, the mating partner is a protective cap including: an enclosure portion enclosing communicating from the leading end to the trailing end for enveloping the injection part, and provided on its trailing end side with a filter capable of discharging air toward the outside and preventing the outflow of the liquid; a bottom face portion extending in the outward direction from the enclosure portion; and a connecting portion formed for enveloping the outer periphery of the outer tube part from the bottom face portion and connected to the outer tube part.

According to at least one embodiment of the invention, moreover, there is provided a connector comprising: a male connector including the connection structure of the transfusion line; and a female connector including: an outer tube having a fitted portion to be fitted in the outer tube part; a septum arranged on the inner circumference side of the outer tube and having a slit formed for inserting the injection part; fixing means for fixing the septum in the outer tube part; a space adjoining the septum and capable of opening the injection part leading end containing the bore portion; an inner tube formed on the inner circumference side of the outer tube part for communicating from the space to the outside; and connection means connected to a discharge side member, into which the liquid discharged from the inner tube flows .

In the connection structure of the transfusion line and the connector having the connection structure, according to at least one embodiment of the invention, the injection part side wall is provided with the opening, in which the protective shutter having a biasing force is disposed on the leading end portion side. The opening is closed, when the connector is not connected, and opened, at a priming time and at the connected time, so that the liquid leakage can be substantially eliminated when the mating member is removed.

Having described the invention in detail, it will be apparent that modifications and variations are possible without departing from the scope of the invention defined in the appended claims.

When introducing elements of the present invention or the illustrated embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

1. A connection structure of a transfusion line having a protective shutter for passing a medical liquid therethrough, comprising: an injection tube including a bottomed tubular injection part having an opening in the leading end side circumferential wall, and an introduction part communicating with the inside of said injection part; a bottomed outer tube part having a bottom portion linked to said injection tube part and opened to the leading end side for fitting a mating partner therein; and a protective shutter externally inserted on said injection part for closing said opening and adapted to move along the axis of said injection part, when it receives a pressing force not lower than a predetermined level toward the base portion side, thereby to open said opening, and to return to said leading end side, when said pressing force is released, thereby to close said opening.

2. A connection structure of a transfusion line having a protective shutter, as claimed in claim 1, wherein said injection part is provided on its base portion side with a taper portion diametrically reduced in the direction to the leading end portion so that said protective shutter is diametrically enlarged along said taper portion, when it slides to the taper portion side, to establish a biasing force toward the leading end portion side.

3. A connection structure of a transfusion line having a protective shutter, as claimed in claim 2, wherein said injection part is compressed to the base portion side in the axial direction, when it slides to the taper portion side, to establish the biasing force toward the leading end portion side.

4. A connection structure of a transfusion line having a protective shutter, as claimed in claim 1, wherein said injection part is provided with a spring portion for biasing said protective shutter to the leading end portion side so that it is biased to the leading end portion side by said spring portion when said protective shutter slides to the spring portion side.

5. A connection structure of a transfusion line having a protective shutter, as claimed in claim 1, wherein said protective shutter is provided with an air-bleeding filter in front of the opening.

6. A connection structure of a transfusion line having a protective shutter, as claimed in claim 1, wherein said mating member is a protective cap that has an enclosure portion enclosing communicating from the leading end to the trailing end for enveloping said injection part, and which is provided on its trailing end side with a filter capable of discharging air toward the outside and preventing the outflow of the liquid; a bottom face portion extending in the outward direction from said enclosure portion; and a connecting portion formed for enveloping the outer periphery of said outer tube part from said bottom face portion and connected to said outer tube part.

7. A connector for a medical liquid flow line, the connector comprising:
i) an injection tube with a closed leading end and a tube wall with a luminal surface and an abluminal surface, there being at least one through hole extending through the tube wall near the closed leading end; and
ii) a shutter that slides on the abluminal surface and occludes the at least one through hole when in a closure disposition, the shutter being displaceable by sliding on the abluminal surface to an open disposition when the shutter is subject to a pressing force when the connector is connected within the flow line, the shutter being biased to the closure disposition so that, when the connector is disconnected from the flow line, the shutter reverts to the closure disposition.

8. A connector comprising:
a male connector including: an injection tube including a bottomed tubular injection part having an opening in the leading end side circumferential wall, and an introduction part communicating with the inside of said injection part; a bottomed outer tube part having a bottom portion linked to said injection tube part and opened to the leading end side for fitting a mating partner therein; and a protective shutter externally inserted on said injection part for closing said opening, the shutter being adapted to move along the axis of said injection part when it receives a pressing force not lower than a predetermined level toward the base portion side to open said opening, and being adapted to return to said leading end side when said pressing force is released to close said opening; and
a female connector including: an outer tube having a fitted portion to be fitted in said outer tube part; a septum arranged on the inner circumference side of said outer tube and made of an elastic member having a slit formed for inserting said injection part; fixing means for fixing said septum in said outer tube part; a space adjoining said septum and capable of opening said injection part leading end containing said bore portion; an inner tube formed on the inner circumference side of said outer tube part for communicating from said space to the outside; and connection means connected to a discharge side member, into which the liquid discharged from said inner tube flows.
